(19) United States
(12) Patent Application Publication
(10) Pub. No.: US 2016/0000493 A1
(43) Pub. Date: Jan. 7, 2016

(54) HIGHLY STABLE GEAR DRIVE ADAPTER

(71) Applicant: Hua Gao, Fox Point, WI (US)

(72) Inventor: Hua Gao, Fox Point, WI (US)

(21) Appl. No.: 14/851,534

(22) Filed: Sep. 11, 2015

Related U.S. Application Data
(63) Continuation-in-part of application No. 14/051,693, filed on Oct. 11, 2013.

(51) Int. Cl. A61B 17/88; B25B 17/00; F16H 1/28

(52) U.S. Cl. CPC A61B 17/8875; F16H 1/28; B25B 17/00; A61B 2017/00407

(57) ABSTRACT

The present invention is a highly stable gear drive adapter featuring a stabilized drive assembly. The drive assembly includes a drive shaft connected to a gear holder assembly, which couples to a gear shaft assembly, which in turn couples to a tool adapter. Each component of the drive assembly stabilizes or is stabilized by another component. A cover aperture and a drive shaft bearing stabilize the drive shaft laterally. Additionally, an interface between a polygonal band on the surface of the drive shaft and a polygonal aperture in the gear holder assembly also stabilizes the drive shaft laterally. A retaining ring and a shaft stop stabilize the drive shaft axially. The gear shaft assembly includes two gear shaft bearings laterally stabilizing a gear shaft and a gear shaft channel that contains the drive shaft bearing. A tool adapter bearing stabilizes the tool adapter.

### CROSS-REFERENCE TO RELATED APPLICATIONS

This application is a continuation-in-part of and claims priority to U.S. patent application Ser. No. 14/051,693, filed on Oct. 11, 2013, which is hereby incorporated by reference in its entirety.

### BACKGROUND

1. Field of Invention

The present invention relates to the field of medical drivers, and more specifically to a gear-driver adapter for medical tools.

2. Description of the Related Art

Drivers are handles used to turn medical implants, screws or other components during surgery. Typical drivers require a complete turn of the handle to complete one turn of the tool shaft. It is a problem known in the art that this motion places significant stress on the surgeon's hand, wrist and arm.

Gear-drivers are mechanisms known in the art that allow surgeons to turn a tool shaft using fewer handle rotations, as expressed by the ratio of the number turns of a tool shaft to the number of actual turns of a handle required by a surgeon. For example, some gear-drivers have a 2:1 gear ratio, requiring only a single turn of the driver to complete 2 turns of the tool shaft. Gear-drivers therefore allow surgeons and patients to spend less time in surgery, and decrease the amount of physical stress on the surgeon.

Another problem known in the art is that subjecting a gear-driver to sudden bursts of torque can destabilize components and decrease the precision of inputs and outputs. Destabilization of components has caused gear drivers to fail during surgery, as documented by FDA incident reports. A second problem with gear-drivers is that both straight drivers and gear-drivers are required to complete a single drive task. A surgeon must therefore manage multiple tools during surgery.

### SUMMARY OF THE INVENTION

One embodiment of the present invention is a highly stable gear drive adapter apparatus including a stabilized drive assembly for transmitting a force. The stabilized drive assembly includes a drive shaft stabilized by a drive shaft bearing, a stabilizing gear holder assembly, a stabilized gear shaft assembly and a tool adapter stabilized by a tool adapter bearing.

Another embodiment of the present invention is a system for stabilizing a drive assembly for a highly stable gear drive adapter including a drive shaft bearing, a tool adapter bearing, a retaining ring and a stabilizing gear holder assembly.

Another embodiment of the present invention is a highly stable gear drive adapter apparatus including a stabilized drive assembly for transmitting a force. The stabilized drive assembly includes a drive shaft stabilized by a drive shaft bearing, a stabilizing gear holder assembly, a stabilized gear shaft assembly and a tool adapter stabilized by a tool adapter bearing. The drive shaft includes a retaining ring groove housing a stabilizing retaining ring. The stabilizing gear holder assembly includes a front stabilizing plate, a back stabilizing plate, a plurality of spacers and a plurality of gears. The stabilized gear shaft assembly includes a gear shaft supported at two points by two gear shaft bearings. The gear shaft has a gear shaft channel that contains at least one drive shaft bearing.

### BRIEF DESCRIPTION OF THE DRAWINGS

FIGS. 1a and 1b illustrate a perspective view and a side view, respectively, of an exemplary gear-driver adapter.

FIGS. 2a and 2b illustrate an exploded view and a cross-sectional view of gear-driver adapter.

FIG. 3 illustrates a front perspective view of a drive shaft.

FIGS. 4a and 4b illustrate a front perspective view and a back perspective view, respectively, of a gear holder assembly.

FIG. 5 illustrates a back perspective view of a gear body.

FIG. 6 illustrates a perspective view of an exemplary gear-driver adapter system in use with an interchangeable handle and a surgical instrument.

### TERMS OF ART

As used herein, the term “adapter” refers to a component of an orthopedic tool handle that engages a tool.

As used herein, the term “gear” refers to any rotating structure having teeth that interact or mesh with another component to transmit torque or motion.

As used herein, the term “stabilize” or “stabilizing” refers to the control of non-utilitarian movement of a component.

### DETAILED DESCRIPTION OF INVENTION

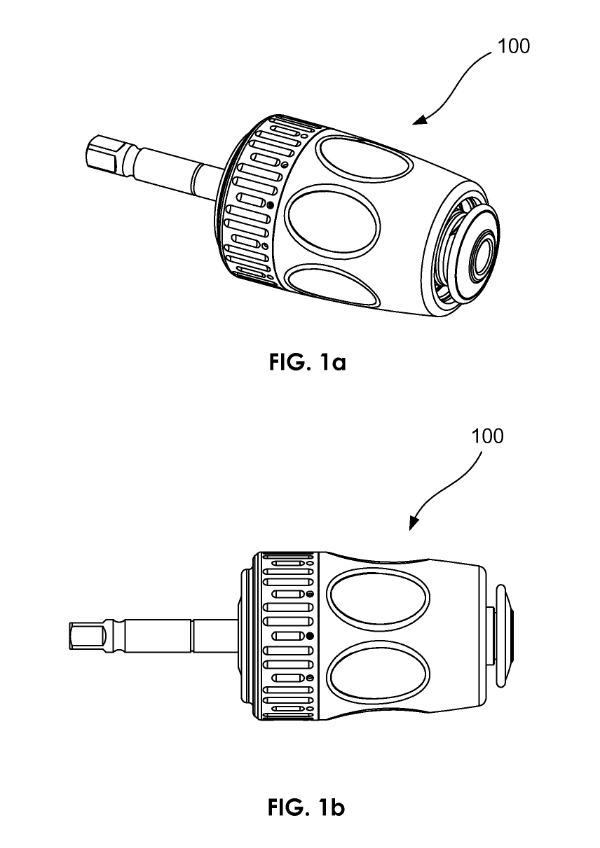

FIGS. 1a and 1b illustrate a perspective view and a side view, respectively, of an exemplary gear-driver adapter 100.

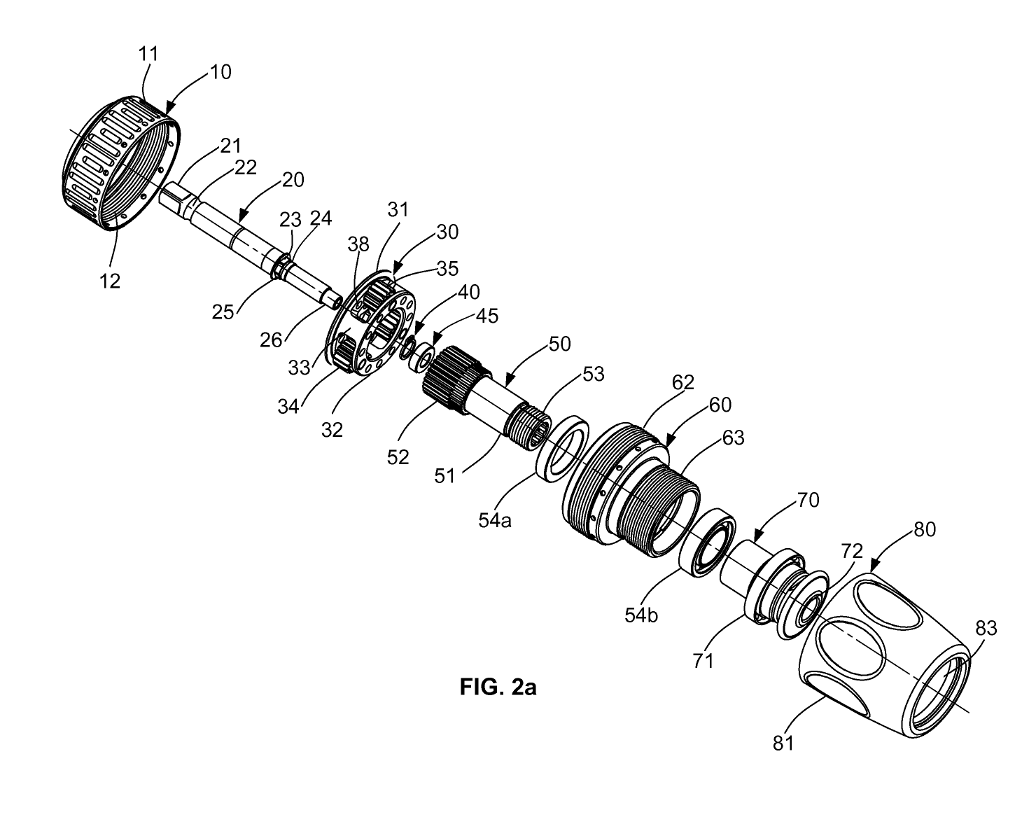

FIGS. 2a and 2b illustrate an exploded view and a cross-sectional view of gear-driver adapter 100. Gear-driver adapter 100 includes a back end having a cover 10, a drive shaft 20, a gear holder assembly 30, a retaining ring 40, a drive shaft bearing 45, a gear shaft assembly 50, a gear body 60, a tool adapter 70 and a gripping handle 80 at a front end. Drive shaft 20, gear holder assembly 30, gear shaft assembly 50 and tool adapter 70 form a stabilized drive assembly.

Cover 10 includes a grooved outer cover surface 11, a threaded inner cover surface 12 and a cover aperture 13. Grooved outer surface 11 may contain any configuration or variety of grooves that facilitate assembly. Threaded inner cover surface 12 provides a threaded connection between cover 10 and gear body 60 for assembly. Cover aperture 13 is sized to accept and provide support to drive shaft 20.

Drive shaft 20 includes handle interface 21, a handle groove 22, a shaft stop 23, a retaining ring groove 24, a polygonal band 25 and a bearing interface 26. In the embodiment shown, handle interface 21 has a square configuration with four flattened surfaces separated by rounded transitions for attachment to a square driver handle. Other embodiments may have a hexagonal, triangular, or any other configuration capable of attaching to a driver handle known in the art. Handle groove 22 can also serve to secure drive shaft 20 to a given driver handle.

When gear-driver adapter 100 is fully assembled, shaft stop 23 and retaining ring groove 24 are found backward and forward, respectively, of drive shaft aperture 37 in gear holder assembly 30. Shaft stop 23 prevents drive shaft 20 from moving forward along its axis. Retaining ring 40, located in retaining ring groove 24, prevents drive shaft 20 from moving backward along its axis. Polygonal band 25 is located within drive shaft aperture 37 in gear holder assembly 30. While the exemplary embodiment of polygonal band 25 shows a hexagonal configuration with six flattened surfaces separated by rounded transitions, other embodiments may have a square, triangular, or any other polygonal configuration.

Bearing interface 26, located at the front end of drive shaft 20, fits together with drive shaft bearing 45 to support drive shaft 20 during use. Because drive shaft 20 has support from cover 10, gear holder assembly 30 and drive shaft bearing 45, it is more stable and less likely to laterally deflect during torque input.

Gear holder assembly 30 includes a back plate 31, a front plate 32, multiple spacers 33, multiple planet gears 34 having planet gear teeth 35, multiple gear axes 36, drive shaft aperture 37 and sterilant apertures 38. In use, gear holder assembly 30 transmits torque or motion from drive shaft 20 to gear shaft 51.

Spacers 33 connect back plate 31 and front plate 32. While the exemplary embodiment shown has three spacers 33, other embodiments may include spacers ranging in number from two to as many as can fit between planet gears 34. Spacers 33 are located equidistantly within the outer perimeters of back plate 31 and front plate 32, but do not extend beyond these edges. Each spacer 33 is located between two planet gears 34.

Each planet gear 34 includes a gear axis 36. In the exemplary embodiment, gear axes 36 are pin-style axes. However, in other embodiments gear axes 36 are any structure or device known in the art to rotationally secure gears 34 within gear holder assembly 30. Because both back plate 31 and front plate 32 provide support to gear axes 36 during use, planet gears 34 have a decreased likelihood of misalignment over the life of gear-driver adapter 100.

Drive shaft aperture 37 is centrally located in back plate 31, and has an inner diameter just larger than the outer diameter of polygonal band 25 of drive shaft 20 to permit insertion of drive shaft 20 within drive shaft aperture 37. The exemplary embodiment of drive shaft aperture 37 shows a hexagonal configuration with six flattened surfaces separated by rounded transitions, corresponding to the exemplary embodiment of polygonal band 25. Other embodiments may have a square, triangular, or any other polygonal configuration in order to accommodate other embodiments of polygonal band 25.

Sterilant apertures 38 are located in back plate 31. These sterilant apertures 38 allow a fluid sterilant to enter into the interior of gear-driver adapter 100 and sterilize it. While the exemplary embodiment includes six sterilant apertures 38, other embodiments may include more or fewer sterilant apertures 38.

Retaining ring 40 interfaces with retaining ring groove 24 to prevent drive shaft 20 from moving backward along its axis. In the exemplary embodiment, retaining ring 40 is a tapered section retaining ring. Other embodiments may utilize constant section retaining rings or spiral retaining rings.

Drive shaft bearing 45 fits together with bearing interface 26 to support drive shaft 20 during use. Because the front end of drive shaft 20 has support from drive shaft bearing 45, it is more stable and less likely to laterally deflect during torque input. In the exemplary embodiment, drive shaft bearing 45 is a rolling-element bearing such as, but not limited to, a ball bearing or a cylinder bearing. Other embodiments may utilize plain bearings such as, but not limited to, a bushing bearing or a two-piece bearing.

Gear shaft assembly 50 includes gear shaft 51, gear shaft teeth 52, gear shaft threading 53, gear shaft bearings 54a and 54b and gear shaft channel 55. Gear shaft 51 transmits torque or motion from gear holder assembly 30 to tool adapter 70.

Gear shaft teeth 52 surround the outer back surface of gear shaft 51. The size of gear shaft teeth 52 corresponds to the size of planet gear teeth 35, allowing gear shaft teeth 52 and planet gear teeth 35 to intermesh. Gear shaft threading 53 surrounds the outer front surface of gear shaft 51 and permits a secure coupling between gear shaft 51 and tool adapter 70.

Gear shaft bearings 54a and 54b are located in front of gear shaft teeth 52 and behind gear shaft threading 53, respectively. Gear shaft bearings 54a and 54b support gear shaft 51 within gear-driver adapter 100 and ensure smooth rotation during use. Gear shaft bearings 54a and 54b also prevent lateral deflection that may cause gear shaft 51 to misalign with gear holder assembly 30 or tool adapter 70. In the exemplary embodiment, gear shaft bearings 54a and 54b are rolling-element bearings such as, but not limited to, a ball bearing or a cylinder bearing. Other embodiments may utilize plain bearings such as, but not limited to, a bushing bearing or a two-piece bearing.

Gear shaft channel 55 extends from a back end of gear shaft 51 to a front end of gear shaft 51. The diameter of gear shaft channel 55 approximately matches the outer diameter of drive shaft bearing 45, allowing positioning of drive shaft bearing 45, and by extension drive shaft 20, within gear shaft 51.

Gear body 60 includes gear body teeth 61, threaded back gear body surface 62, threaded front gear body surface 63 and gear body channel 64. Gear body teeth 61 surround the inner back surface of gear body 60. The size of gear body teeth 61 corresponds to the size of planet gear teeth 35, allowing gear body teeth 61 and planet gear teeth 35 to intermesh.

In the exemplary embodiment shown, the inner surface of gear body 60, the outer surfaces of planet gears 34 and the outer surface of gear shaft 51 act as a planetary gearing system in which the inner surface of gear body 60 is an annulus or ring, gear holder assembly 30 is a planet carrier for planet gears 34 and the outer surface of gear shaft 51 is the central, or sun, gear. In the planetary gearing system described, the inner surface of gear body 60, or the annulus or ring, holds stationary, with input rotation provided by planet gears 34. The outer surface of gear shaft 51, or sun gear, produces the output rotation, which is communicated to tool adapter 70 through its coupling.

In the exemplary embodiment described, this arrangement results in a fixed gear ratio, meaning the outer surface of gear shaft 51 is the central, or the sun gear, rotates a given number of times for each rotation of planet gears 34. In further exemplary embodiments, gear body 60, planet gears 34 and gear shaft 51 may be of different sizes or have different numbers of teeth. In still further exemplary embodiments, gear holder assembly 30 may include more or fewer planet gears 34. The gear ratio of gear-drive adapter 100 may be increased or decreased by modifying these factors.

Threaded back gear body surface 62 couples gear body 60 to cover 10 through threaded inner cover surface 12. Threaded front gear body surface 63 couples gear body 60 to gripping handle 80. However, in further exemplary embodiments, coupling gear body 60 to cover 10 and/or gripping handle 80 may occur through any method known in the art, including clips, snap-fits, contours, and other structures. Gear body channel 64 supports gear shaft bearings 54a and 54b.

Tool adapter 70 includes tool adapter bearing 71 and tool adapter aperture 72. Tool adapter 70 is an internal adapter having a release collar, as known in the art. However, further exemplary embodiments may use a different tool adapter known in the art. Tool adapter bearing 71 supports tool adapter 70 during torque output. In the exemplary embodiment, tool adapter bearing 71 is a rolling-element bearing such as, but not limited to, a ball bearing or a cylinder bearing. Other embodiments may utilize plain bearings such as, but not limited to, a bushing bearing or a two-piece bearing. Because tool adapter 70 has support from tool adapter bearing 71, it is more stable and less likely to laterally deflect during torque output. Tool adapter aperture 72 may be configured to receive any tool known in the art.

Gripping handle 80 includes contoured outer gripping handle surface 81, threaded inner gripping handle surface 82 and gripping handle aperture 83. In the exemplary embodiment, outer gripping handle surface 81 is a contoured surface. In further exemplary embodiments, outer gripping handle surface 81 may contain any variety of contours and be made of any material which facilitates gripping and provides a comfortable and stable grip. Threaded inner gripping handle surface 82 couples gripping handle 80 to gear body 60. Gripping handle aperture 83 permits passage of tool adapter aperture 72 during assembly.

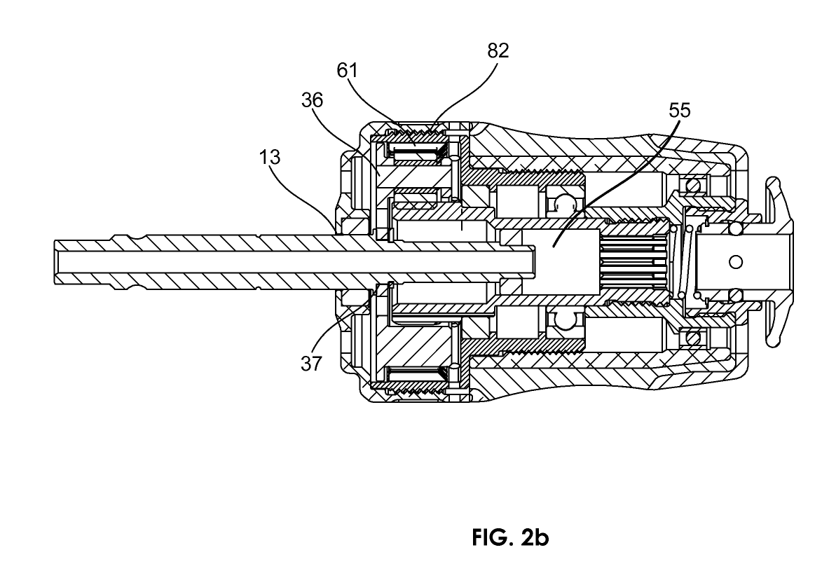

FIG. 3 illustrates a front perspective view of drive shaft 20.

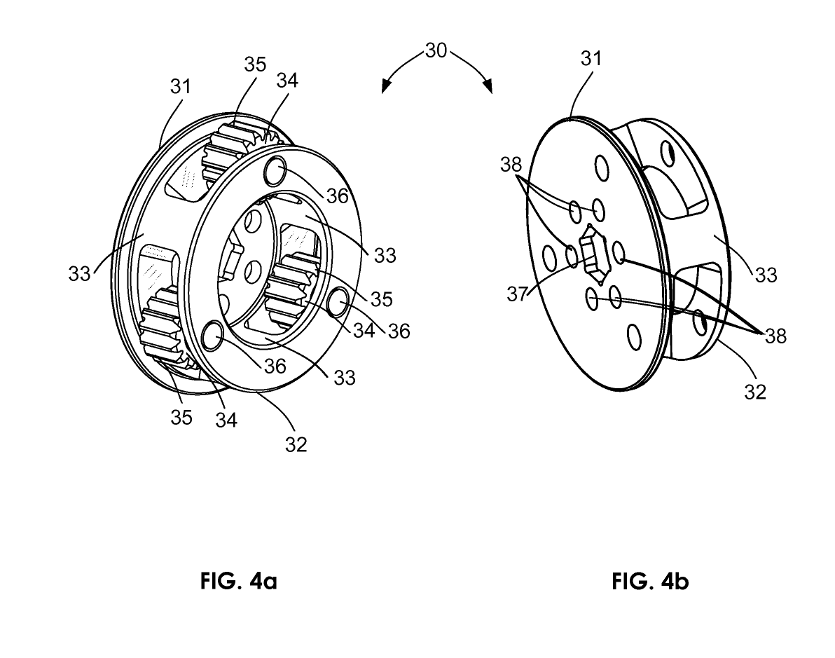

FIGS. 4a and 4b illustrate a front perspective view and a back perspective view, respectively, of gear holder assembly 30. Planet gears 34 and gear axes 36 are not shown in FIG. 4b for clarity.

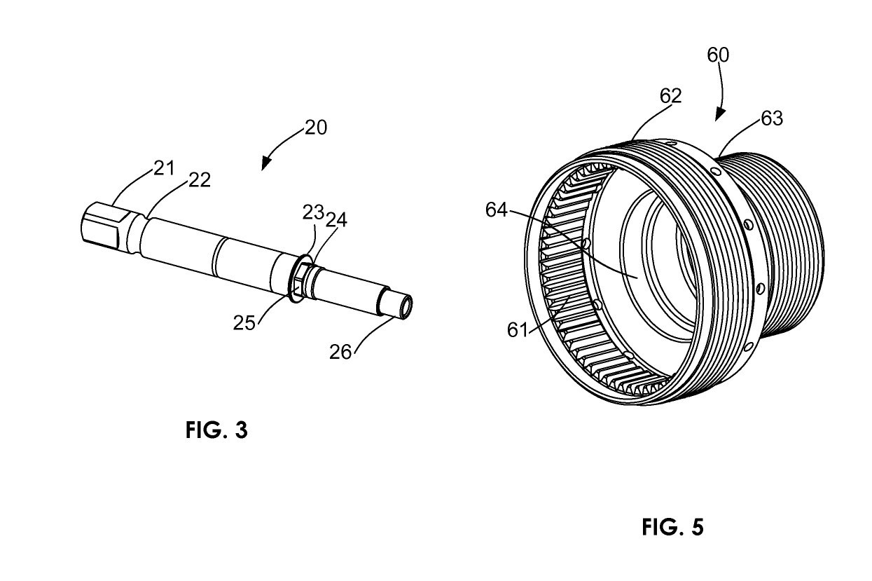

FIG. 5 illustrates a back perspective view of gear body 60.

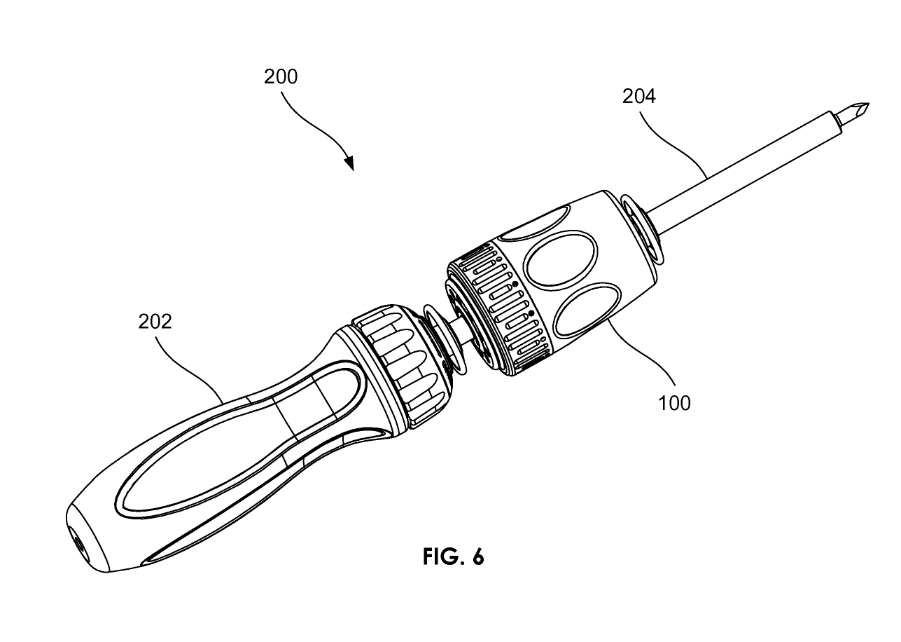

FIG. 6 illustrates a perspective view of an exemplary gear-driver adapter system 200 in use with an interchangeable handle 202 and a surgical instrument 204. Gear-driver adapter system 200 features interchangeable handle 202, which is adapted to connect securely to gear-driver adapter 100. In the embodiment shown, interchangeable handle 202 and gear-driver adapter 100 have corresponding mechanical engagements. In the embodiment shown, the engagements are on drive shaft 20 and the interior surface of component of interchangeable handle 202 (not shown). The mechanical engagements may conform to specifications for the corresponding geometry. In the embodiment shown, drive shaft 20 interfaces with interchangeable handle 202 at handle interface 21. Surgical tool 204 attaches at the front portion of gear-driver adapter 100 at tool adapter aperture 72.

In the exemplary embodiment, interchangeable handle 202 may be rotated in either direction, resulting in rotational movement of surgical tool 204 in the same direction. In further exemplary embodiments, rotation of interchangeable handle 202 may be turned in only one direction. In still further exemplary embodiments, interchangeable handle 202 may be rotated in either direction while surgical tool 204 is configured to turn in only one direction.

In the exemplary embodiment shown, gear-driver adapter 100 operates at a fixed gear ratio. In one embodiment, gear-driver adapter 100 operates at a 1:1 gear ratio, and a single turn of interchangeable handle 202 causes a single turn of surgical tool 204. In another embodiment, gear-driver adapter 100 operates at a 3:1 gear ratio, and a single turn of interchangeable handle 202 causes three complete turns of surgical tool 204.

In further exemplary embodiments, gear-driver adapter 100 may be configured to operate at any gear ratio, although the range of gear ratios that may be used with gear-driver adapter 100 may be limited by reasonable size constraints of interchangeable handle 202, gear-driver adapter 100 and surgical tool 204. In most exemplary embodiments, gear-driver adapter 100 may be configured to operate at gear ratios between 1:5 and 8:1, where a 1:5 ratio provides one complete rotation of surgical tool 204 for every five rotations of interchangeable handle 202 and an 8:1 ratio provides eight complete rotations of surgical tool 204 for every one rotation of interchangeable handle 202.

It will be understood that many additional changes in the details, materials, procedures and arrangement of parts, which have been herein described and illustrated to explain the nature of the invention, may be made by those skilled in the art within the principle and scope of the invention as expressed in the appended claims.

It should be further understood that the drawings are not necessarily to scale; instead, emphasis has been placed upon illustrating the principles of the invention. Moreover, the term “approximately” as used herein may be applied to modify any quantitative representation that could permissibly vary without resulting in a change in the basic function to which it is related.

What is claimed is:

1. A highly stable gear drive adapter apparatus comprised of:
a stabilized drive assembly for transmitting a force, wherein said stabilized drive assembly is comprised of:
a drive shaft stabilized by a drive shaft bearing;
a stabilizing gear holder assembly;
a stabilized gear shaft assembly; and
a tool adapter stabilized by a tool adapter bearing.

2. The apparatus of claim 1, wherein said drive shaft further comprises a retaining ring groove housing a stabilizing retaining ring.

3. The apparatus of claim 2, wherein said retaining ring is selected from a group consisting of: tapered section retaining rings, constant section retaining rings and spiral retaining rings.

4. The apparatus of claim 2, wherein said drive shaft bearing is located at a front end of said drive shaft and said retaining ring is located backwards of said drive shaft bearing.

5. The apparatus of claim 2, wherein said drive shaft further comprises a shaft stop located backwards of said retaining ring groove.

6. The apparatus of claim 1, wherein said stabilizing gear holder assembly is comprised of:
a front stabilizing plate;
a back stabilizing plate;
a plurality of spacers; and
a plurality of gears.

7. The apparatus of claim 6, wherein said back stabilizing plate comprises a central polygonal aperture corresponding to a polygonal band surrounding a section of said drive shaft.

8. The apparatus of claim 6, wherein said plurality of spacers are located between and equidistantly around a perimeter of said back stabilizing plate and said front stabilizing plate.

9. The apparatus of claim 6, wherein each of said plurality of spacers is located between two of said plurality of gears.

10. The apparatus of claim 6, wherein each of said plurality of gears comprises a gear axis supported at a first end by said back stabilizing plate and at a second end by said front stabilizing plate.

11. The apparatus of claim 1, wherein said stabilized gear shaft assembly comprises a gear shaft supported at two points by two gear shaft bearings.

12. The apparatus of claim 1, wherein said stabilized gear shaft assembly comprises a gear shaft having a gear shaft channel, wherein said drive shaft bearing is contained within said gear shaft channel.

13. The apparatus of claim 12, wherein said gear shaft stabilizes drive shaft and said tool adapter.

14. The apparatus of claim 1, wherein said tool adapter bearing and said drive shaft bearing are selected from the group consisting of: rolling-element bearings and plain bearings.

15. The apparatus of claim 1, further comprised of a cover, wherein said cover comprises a cover aperture sized to accept and provide support to said drive shaft.

16. The apparatus of claim 1, further comprised of a gripping handle, wherein said gripping handle comprises a contoured outer gripping handle surface manufactured from silicone, wherein said gripping handle is hollow to reduce weight.

17. The apparatus of claim 1, wherein portions of said stabilized drive assembly are constructed from a non-steel material to reduce weight.

18. The apparatus of claim 1, wherein said force is a rotational force.

19. A system for stabilizing a drive assembly for a highly stable gear drive adapter, comprised of:
a drive shaft bearing;
a tool adapter bearing;
a retaining ring; and
a stabilizing gear holder assembly.

20. A highly stable gear drive adapter apparatus comprised of:
a stabilized drive assembly for transmitting a force, wherein said stabilized drive assembly is comprised of:
a drive shaft stabilized by a drive shaft bearing, wherein said drive shaft further comprises a retaining ring groove housing a stabilizing retaining ring;
a stabilizing gear holder assembly, wherein said stabilizing gear holder assembly is comprised of a front stabilizing plate, a back stabilizing plate, a plurality of spacers, and a plurality of gears;
a stabilized gear shaft assembly comprising a gear shaft supported at two points by two gear shaft bearings, said gear shaft having a gear shaft channel, wherein at least one of said drive shaft bearing is contained within said gear shaft channel; and
a tool adapter stabilized by a tool adapter bearing.
